(19) United States
(12) Patent Application Publication
(10) Pub. No.: US 2012/0041342 A1
(43) Pub. Date: Feb. 16, 2012

(54) HIGH DURABILITY CORONARY GUIDE WIRE

(75) Inventor: Raleigh A. Purtzer, Winchester, CA (US)

(73) Assignee: ABBOTT CARDIOVASCULAR SYSTEMS INC., Santa Clara, CA (US)

(21) Appl. No.: 12/857,149

(22) Filed: Aug. 16, 2010

(51) Int. Cl. A61M 25/09

(52) U.S. Cl. 600/585

(57) ABSTRACT

A guide wire for intra-luminal advancement of a medical device within a patient has an elongate core member with a flexible body member disposed on a distal section. The distal tip of the guide wire is cold worked by reducing the cross-sectional area of the distal tip so that the superelastic properties of the distal tip are transformed to pseudoelastic properties thereby allowing the distal tip to be more shapeable.

### FIELD OF THE INVENTION

This invention relates generally to the field of medical devices, and more particularly to a guide wire for advancing a procedure such as percutaneous transluminal coronary angioplasty (PTCA) or stent.

### BACKGROUND

Conventional guide wires for angioplasty and other vascular procedures usually comprise an elongated core member with one or more tapered sections near the distal end thereof and a flexible body such as a helical coil disposed about the distal portion of the core member. A shapeable member, which may be the distal extremity of the core member or a separate shaping ribbon which is secured to the distal extremity of the core member, extends through the flexible body and is secured to a rounded plug at the distal end of the flexible body. Torquing means are provided on the proximal end of the core member to rotate, and thereby steer, the guide wire while it is being advanced through a patient's vascular system.

In a typical PTCA procedure, a guiding catheter having a preformed distal tip is percutaneously introduced into the cardiovascular system of a patient in a conventional Seldinger technique and advanced therein until the distal tip of the guiding catheter is seated in the ostium of a desired coronary artery. A guide wire is positioned within an inner lumen of a dilatation catheter and then both are advanced through the guiding catheter to the distal end thereof. The guide wire is first advanced out of the distal end of the guiding catheter into the patient's coronary vasculature until the distal end of the guide wire crosses a lesion to be dilated, then the dilatation catheter having an inflatable balloon on the distal portion thereof is advanced into the patient's coronary anatomy over the previously introduced guide wire until the balloon of the dilatation catheter is properly positioned across the lesion. Once in a position across the lesion, the balloon is inflated to a predetermined size with radiopaque liquid at relatively high pressures (e.g., greater than 4 atmospheres) to press the arteriosclerotic plaque of the lesion against the inside of the artery wall and to otherwise expand the inner lumen of the artery. The balloon is then deflated so that blood flow is resumed through the dilated artery and the dilatation catheter can be removed therefrom.

A major requirement for guide wires is that they have sufficient column strength to be pushed through a patient's vascular system or other body lumen without kinking. However, they must also be flexible enough to avoid damaging the blood vessel or other body lumen through which they are advanced. Efforts have been made to improve both the strength and flexibility of guide wires to make them more suitable for their intended uses, but these two properties are for the most part diametrically opposed to one another in that an increase in one usually involves a decrease in the other.

Conventional guide wires for angioplasty, stent delivery, atherectomy and other intravascular procedures usually comprise an elongate core member with one or more tapered segments near the distal end thereof. A flexible body member, such as a helical coil or a tubular body of polymeric material, is typically disposed about the distal portion of the core member. A shapeable member, which may be the distal extremity of the core member or a separate shapeable ribbon which is secured to the distal extremity of the core member extends through the flexible body and is secured to the distal end of the flexible body by soldering, brazing or welding, or an adhesive in the case of polymeric flexible bodies which forms a rounded distal tip. The leading tip is highly flexible and will not damage or perforate the vessel and the portion behind the distal tip is increasingly stiff which better supports a balloon catheter or similar device.

The shapeable member or ribbon of a typical guide wire is a small diameter wire which has been flattened to a constant transverse profile. Flattening of the shapeable member facilitates the shapeability of the member, however, a shapeable member having a constant transverse profile or flexibility can be subject to prolapse during use. Prolapse occurs when the shapeable member gets bent back on itself in a constrained lumen and is difficult to straighten out with proximal manipulation. One method of preventing prolapse or reducing the occurrence thereof is to have increased stiffness at a proximal end of a shapeable member. This has been done with incremental steps in the shapeable member with thinner more flexible steps distally creating greater flexibility distally. However, the use of incremental steps can cause abrupt changes in flexibility of the shapeable member which can be detrimental to smooth tracking and performance of the guide wire. What has been needed is a guide wire with a shapeable member at the distal section that is flexible and maintains a shapeable character. The present invention satisfies these and other needs.

### SUMMARY OF THE INVENTION

The present invention is directed to a guide wire having an elongate core member with a proximal section and a distal section. The distal section preferably has at least one flexible segment that is tapered and initially has a circular transverse cross-section. The distal tip is formed into a flattened oval section and it is cold worked. The flattened oval has two or more opposed faces that are parallel or distally tapered in essentially the same or mirror image relationship to each other. Prior to cold working, the distal tip is formed from a metal alloy having superelastic properties, and after cold working the distal tip has pseudoelastic properties, which provides superior performance as a shapeable member. The proximal section of the elongate core member is nominally round or circular in cross-section and preferably with a constant diameter and the round cross-section preferably extends distally to a proximal end of the flexible segment disposed on the distal section of the core. The flexible segment may be disposed anywhere on the distal section, but preferably is disposed at a distal end of the distal section, serving as a shapeable member.

In one embodiment, a guide wire has an elongated core section having a distal end and a proximal end. The distal end has a substantially circular or round cross-section prior to being cold worked, and a substantially flattened oval section after being cold worked. In this embodiment, a 0.003 inch diameter wire is flattened to 0.00175 inch during cold working. The resulting distal tip is shapeable and resistant to kinking during use. The guide wire is formed from a shape memory alloy such as nitinol. After cold working, the distal tip has pseudoelastic properties so that the tip can be bent or shaped with finger pressure. In one embodiment, after cold working and forming the distal tip into a flattened oval cross-section, the distal tip is coated in gold to increase solderability. In this embodiment, the distal tip is cold worked with the amount of cold reduction in the range of about 33% to 46%.

These and other advantages of the invention will become more apparent from the following detailed description of the preferred embodiments when taken in conjunction with the accompanying exemplary drawings.

### BRIEF DESCRIPTION OF THE DRAWINGS

FIG. 1 is an elevational view, partially in section, of a guide wire embodying features of the invention.

FIG. 2 is an enlarged elevational view of a portion of the distal section of the elongated core member of the guide wire shown in FIG. 1.

FIG. 3 is a transverse cross-sectional view taken along lines 3-3 depicting a substantially circular or round cross-section of the distal end of the guidewire.

FIG. 4 is an enlarged elevational view of a portion of the distal section of the elongated core member of the guide wire with a pair of rollers compressing the distal end of the guide wire.

FIG. 5 is a transverse cross-sectional view taken along lines 5-5 depicting a flattened oval cross-section of the distal end of the guide wire after being passed through compression rollers.

FIG. 6 is an elevational view of a portion of the distal section of the elongated core member.

FIG. 7 is a transverse cross-sectional view taken along lines 7-7 depicting a substantially circular or round cross-section of the distal end of the guide wire.

FIG. 8 is an enlarged elevational view of a portion of the distal section of the elongated core member depicting a pair of compression rollers compressing the distal end of the guide wire.

FIG. 9 is a transverse cross-sectional view taken along lines 9-9 depicting a flattened oval cross-section of the distal end of the guide wire.

FIG. 10 is an enlarged elevational view of a portion of the distal section of the elongated core member in which a pair of relief dies compress the distal end of the guide wire.

FIG. 11 is a transverse cross-sectional view of the distal end of the guide wire of FIG. 10 depicting a flattened oval cross-section of the distal end after the relief dies have compressed the distal end of the guide wire.

### DETAILED DESCRIPTION OF THE PREFERRED EMBODIMENTS

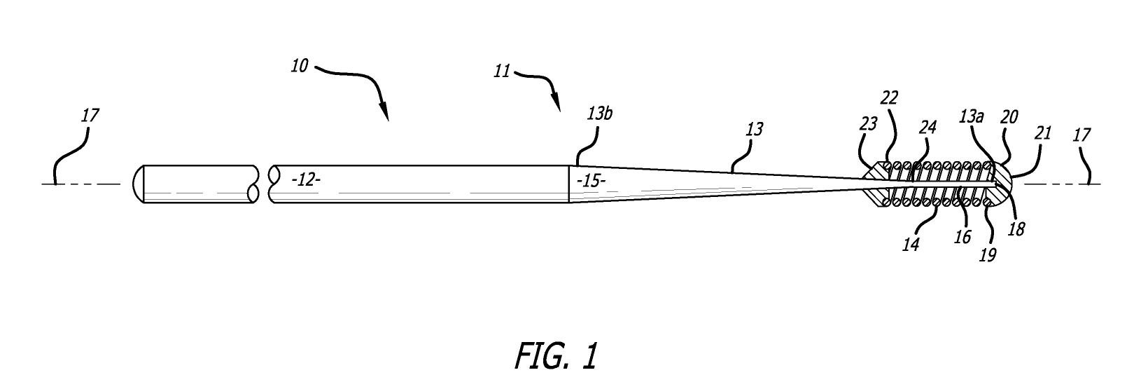

FIG. 1 is an elevational view of guide wire 10 which embodies features of the invention, and which includes an elongated core member 11 with a proximal core section 12, a distal core section 13, and a flexible body member 14 which is fixed to the distal core section. The distal core section 13 has a tapered segment 15, and a flexible segment 16 which is distally contiguous to the tapered segment 15. The distal section 13 may also have more than one tapered segment 15 which have typical distally decreasing tapers with substantially round or circular transverse cross-sections. A longitudinal centerline 17 extends through the elongated core member 11.

The core member 11 is optionally coated with a lubricious coating such as a fluoropolymer, e.g. TEFLON® available from DuPont, which extends the length of the proximal core section. The distal section 13 is also provided with a lubricious coating, such as MICROGLIDE™ coating used by the present assignee, Abbott Cardiovascular Systems Inc. on many of its commercially available guide wires. Hydrophilic coatings may also be employed.

The length and diameter of guide wire 10 may be varied to suit the particular procedures in which it is to be used and the materials from which it is constructed. The length of the guide wire 10 generally ranges from about 65 cm to about 320 cm, more typically ranging from about 160 cm to about 200 cm, and preferably from about 175 cm to about 190 cm for the coronary anatomy. The guide wire diameter generally ranges from about 0.008 inch to about 0.035 inch (0.203 to 0.889 mm), more typically ranging from about 0.012 inch to about 0.018 inch (0.305 to 0.547 mm), and preferably about 0.014 inch (0.336 mm) for coronary anatomy.

The flexible segment 16 terminates in a distal end 18. Flexible body member 14, preferably a helical coil, surrounds a portion of the distal section 13 of the elongated core member 11, with a distal end 19 of the flexible body member 14 secured to the distal end 18 of the flexible segment 16 by a body of solder 20. The proximal end 22 of the flexible body member 14 is similarly bonded or secured to the distal core section 13 by a body of solder 23. Materials and structures other than solder may be used to join the flexible body 14 to the distal core section 13, and the term “body of solder” includes other materials such as braze, epoxy, polymer adhesives, including cyanoacrylates and the like.

The flexible segment 16 has a length typically range of about 1 cm to about 12 cm, preferably about 2 cm to 10 cm, although longer sections may be used. The form of taper of the flexible segment 16 provides a controlled longitudinal variation and transition in flexibility (or degree of stiffness) of the core segment. The flexible segment is contiguous with the core member 11 and is distally disposed on the distal section 13 so as to leave a shapeable member.

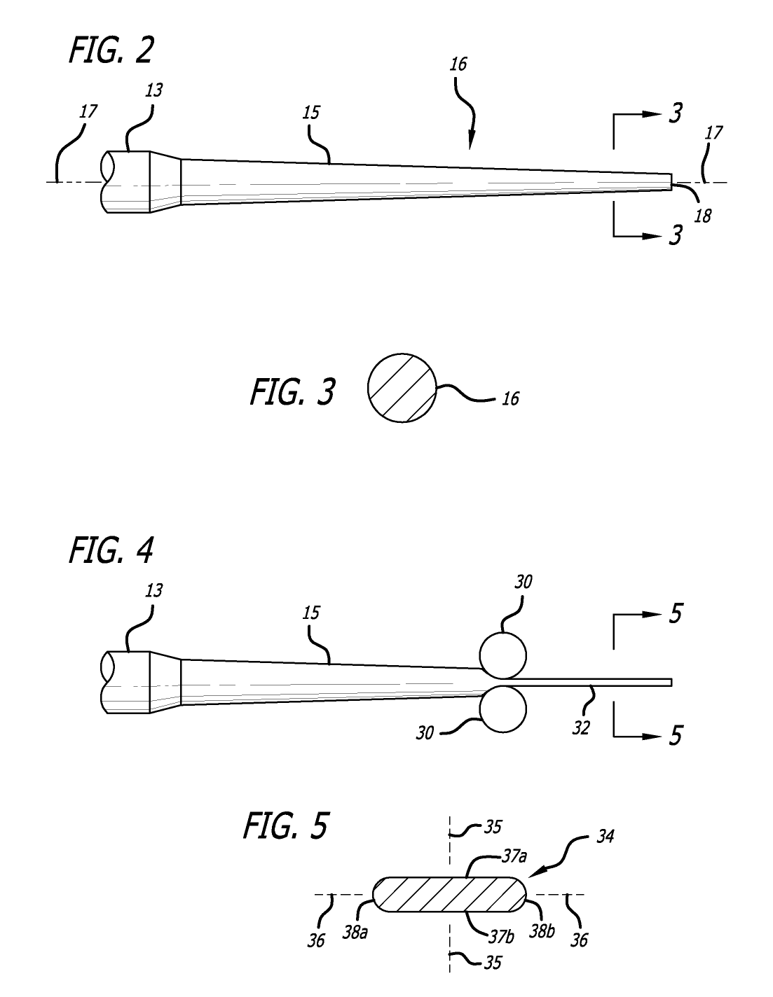

In keeping with the invention, as shown in FIGS. 2-5, the distal core section 13 has a tapered segment 15 as previously described. The transverse cross-section of tapered segment 15 is circular or round as shown in FIG. 3. In order to transform the tapered segment 15 into a shapeable member, the tapered segment is cold worked in order to transform the superelastic material into a pseudoelastic material. Several forms of cold working the flexible segment can be used to transform the superelastic material into pseudoelastic material. For example, as shown in FIG. 4, the tapered segment 15 is fed through a pair of rollers 30 which compresses the tapered segment to form shapeable segment 32. As shown in FIG. 5, the transverse cross-sectional configuration after passing through rollers 30 is a flattened oval 34. The flattened oval 34 has a minor axis 35 and a major axis 36. Further, the flattened oval 34 has a first curved surface 37a and a second curved surface 37b, and a first curved edge 38a and a second curved edge 38b. In one embodiment, the diameter of the flexible segment 16 as shown in FIG. 3 is 0.0028 inch before passing through the rollers, and the diameter of the minor axis 34 (in FIG. 5) is 0.0010 inch after passing through rollers 30 to form the flattened oval 34. In another embodiment, the diameter as shown in FIG. 5 is 0.0028 inch before passing through the rollers, and is 0.0016 inch along the minor axis 34 after passing through the rollers to form the flattened oval configuration. It is preferred that the flexible segment 16 passes through the rollers only one time in order to cold work the material and transform it from a superelastic material to a pseudoelastic material. Preferably, when passing through the rollers 30, the material is not transformed into a linear elastic material, which is too stiff and may kink when shaped by the physician during use. Importantly, the pseudoelastic shapeable segment 32 should have good shape retention properties, resist prolapse, and be easily shapeable using finger pressure by the physician during use. The length of shapeable segment 32 can vary from 0.10 inch to 0.75 inch.

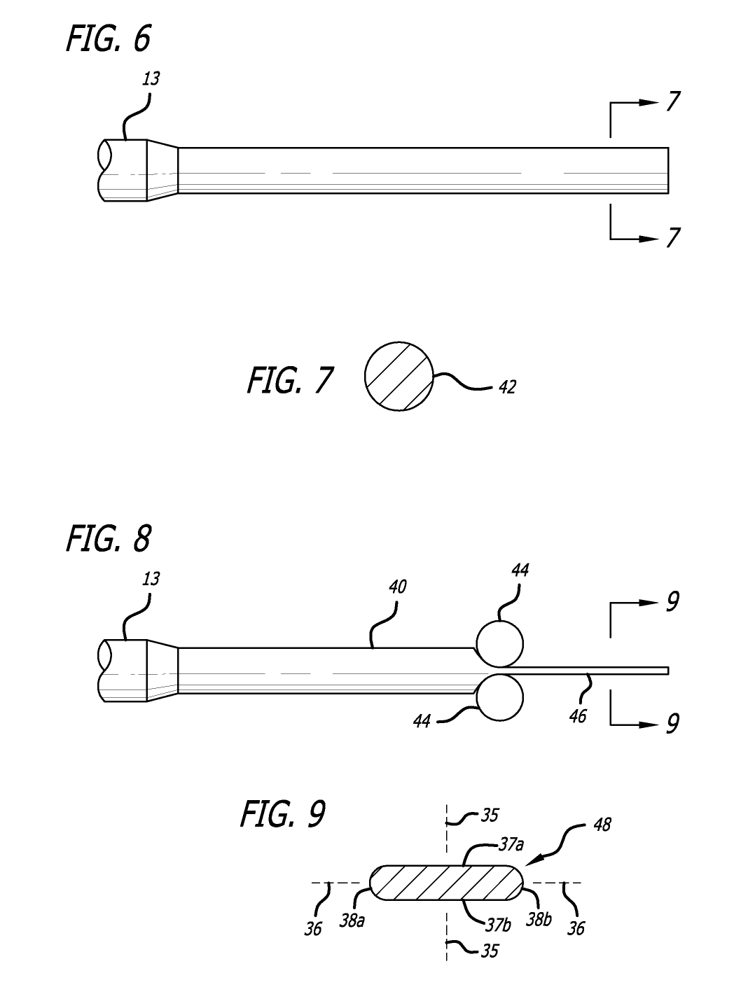

In another embodiment as shown in FIGS. 6-9, the distal core section 13 has a distal end 40 that has a transverse cross-section 42 that is substantially round or circular as shown in FIG. 7. As shown in FIGS. 8-9, the distal end is passed through a pair of rollers 44 and the distal tip 46 is reduced in cross-section to form the flattened oval 48 as shown in FIG. 9. The dimensions are similar to those described for FIGS. 2-5. Again, it is intended that the superelastic material be transformed by cold working through the rollers 44 to form a pseudoelastic material that is easily shapeable by the physician, yet is strong enough to resist prolapse and maintain the shape retention during use. One or more passes through rollers 44 may be required to reduce the cross-sectional area of the flexible segment 16 to form flattened oval 48. Care must be taken, however, so that the cold working does not transform the superelastic material into linear elastic material which may be too stiff for use as the distal tip 46 of the guide wire.

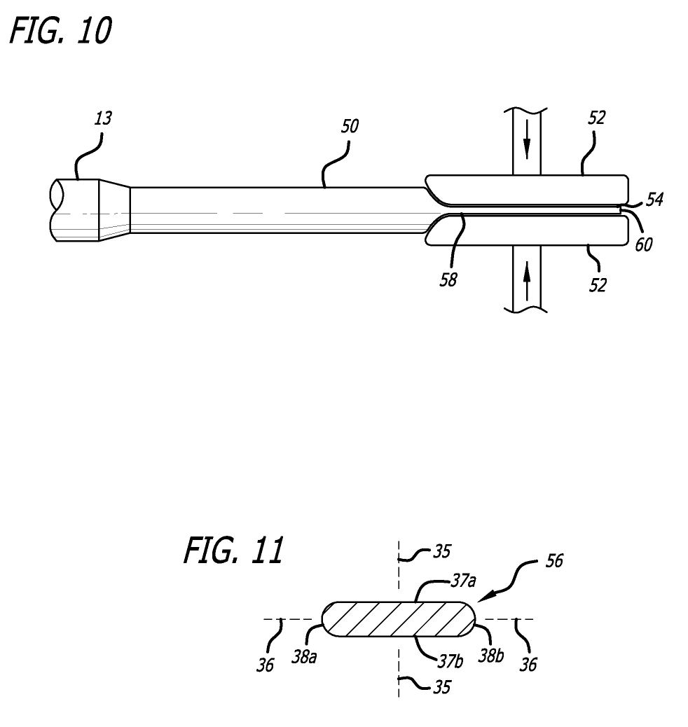

In another embodiment, as shown in FIGS. 10 and 11, a distal core section 13 is similar to that shown in FIG. 6 prior to being cold worked. In this embodiment, distal end 50 is cold worked by using an impact flattener using relief dies 52 and applying one or more impact strikes to form distal tip 54. The transverse cross-section of distal tip 54 is the flattened oval 56 shown in FIG. 11. Again, the relief dies provide sufficient cold working pressure in flattening the distal tip 54 to transform the superelastic material to pseudoelastic material. In one preferred embodiment, a single strike is used on the relief dies to shape the distal tip into the flattened oval 56. More impact strikes may be required in order to achieve the desired result, however, it is preferred that the material not be transformed into linear elastic material as previously described. In this embodiment, the distal tip 54 can have a length from approximately 0.10 inch to about 0.75 inch. Further, the relief dies 52 can be configured so that the distal tip 54 tapers gradually from a thicker flattened oval 56 at the proximal end 58 of the distal tip to a thinner flattened oval at the distal end 60 of the distal tip. In this embodiment, the diameter of the distal end 50 prior to cold working is approximately 0.003 inch which is flattened to the flattened oval 56 having a diameter on the minor axis 34 of 0.00175 inch (a ratio of about 7/12). This ratio provides good transformation from superelastic to pseudoelastic material and provides a shapeable segment that is easily bent or shaped by the physician using finger pressure and will resist prolapse and retain the shape during use. In one embodiment, the diameter of the distal end 50 prior to cold working is in the range from 0.0042 inch to 0.0018 inch, and the minor axis 35 diameter after compression of the distal end by the relief dies is in the range from 0.0024 inch to 0.0010 inch.

With respect to the disclosed embodiments, a guide wire having a core member 11 with a diameter that is larger than those disclosed could be cold worked and flattened into a flattened oval as previously described. In this embodiment, the first and second curved edges 38a,38b, could be die cut and ground so that the distal end of the guide wire would have a more rounded shape rather than a flattened oval. The grinding operation is known in the art and typically grinders are used with liquid cooling/lubricant flowing on the ground distal end to remove the first and second curved edges without appreciably raising the temperature or disturbing the transition from superelastic material to pseudoelastic material.

With respect to each of the disclosed embodiments, the distal tip or flattened oval section is approximately the thickness of a human hair and is very delicate. The distal tip typically is coated in gold to increase its ability to be soldered to a flexible body member 14 (coil) as previously described.

With respect to all of the disclosed embodiments, the guide wire 10 can be formed of a superelastic material such as nitinol. It may be beneficial, however, to provide the elongated core member 11 with a proximal core section 12 that is formed of stainless steel wire, with the distal core member 13 being formed of a superelastic material such as nitinol. Each embodiment has its advantages and is selected to provide a particular need.

While particular forms of the invention have been presented and described, it will be apparent that various modifications can be made without departing from the spirit and scope of the invention. Accordingly, it is not intended that the invention be limited, except as by the appended claims.

1. A guide wire, comprising:
an elongated core section having a distal end and a proximal end;
the distal end having a distal tip that has a circular cross-section prior to being cold worked and a flattened oval cross-section after being cold worked.

2. The guide wire of claim 1, wherein the elongated core section is formed from a superelastic material and the cold worked substantially flattened oval cross-section is transformed from the superelastic material to a pseudoelastic material.

3. The guide wire of claim 1, wherein the distal tip having been cold worked with an amount of cold reduction in the range from 33% to 46%.

4. The guide wire of claim 1, wherein the distal tip is cold worked by relief dies compressing the distal tip to form the pseudoelastic material.

5. The guide wire of claim 1, wherein the distal tip has a diameter of 0.0030 inch before cold working and a diameter of a minor axis of the flattened oval section of 0.00175 inch after cold working.

6. The guide wire of claim 1, wherein the distal tip has a diameter in the range from 0.0042 inch to 0.0018 inch before cold working, and a minor axis diameter of the flattened oval section in the range from 0.0024 inch to 0.0008 inch.

7. The guide wire of claim 1, wherein the distal tip has a length in the range from 0.10 inch to 0.75 inch.

8. The guide wire of claim 1, wherein the guide wire is formed from a superelastic material.

9. The guide wire of claim 8, wherein the guide wire maintains the superelastic material before and after cold working the distal tip, while the distal tip transforms from the superelastic material to a pseudoelastic material after cold working.

10. The guide wire of claim 1, wherein the distal tip is coated in gold.

11. The guide wire of claim 1, wherein the flattened oval cross-section has edges, the edges being removed and the circular cross-section of the distal end of the guide wire and the flattened oval cross-section are ground down to form a smooth transition section.

12. The guide wire of claim 11, wherein after the grinding operation, the distal end of the guide wire remains superelastic material and the distal tip remains pseudoelastic material.

13. The guide wire of claim 1, wherein the flattened oval cross-section has a first curved surface and an opposed second curved surface and a pair of curved edges.

14. A method for forming a shapeable tip on a guide wire, comprising:
providing an elongated core section having a distal end and a proximal end, cold working the distal end by compressing the distal end and transforming a circular cross-section into a flattened oval cross-section to form a shapeable tip.

15. The method of claim 14, wherein the elongated core section is formed from a superelastic material and the cold worked substantially flattened oval cross-section is transformed from the superelastic material to a pseudoelastic material.

16. The method of claim 14, wherein cold working the shapeable tip with an amount of cold reduction in the range from 33% to 46%.

17. The guide wire of claim 14, wherein cold working the shapeable tip by using relief dies to compress the shapeable tip to form the pseudoelastic material.
